(19) United States
(12) Patent Application Publication
(10) Pub. No.: US 2009/0197327 A1
(43) Pub. Date: Aug. 6, 2009

(54) Culturing Bag

(75) Inventor: Reinhard Bergmann, Wallenhorst (DE)

Correspondence Address: GUDRUN E. HUCKETT DRAUDT, SCHUBERTSTR. 15A, WUPPERTAL, 42289, DE

(73) Assignee: BISCHOF + KLEIN GMBH & CO. KG, Lengerich (DE)

(21) Appl. No.: 12/362,497

(22) Filed: Jan. 30, 2009

(30) Foreign Application Priority Data
Feb. 1, 2008 (DE) 20 2008 001 453.0

(51) Int. Cl. C12M 1/00

(52) U.S. Cl. 435/297.1

(57) ABSTRACT

A culturing bag for microorganisms has a wall formed of plastic film wherein at least one wall area of the wall is permeable for oxygen and carbon dioxide. A first layer of microporous film is attached to the at least one wall area as an exterior layer so as to form a multi-layer configuration in the at least one wall area. The first layer of microporous film is sealed by a seal-tight edge seam to the wall. The microporous film is adapted to be torn open or torn off. At least one second layer of microporous film can be placed between the exterior layer and the wall.

The invention concerns a culturing bag for microorganisms as disclosed in DE-OS 26 15 680, for example. Such culturing bags for microorganisms are generally to be embodied to be sterilizable, moisture-resistant, and non-rotting in order to enable the development of microorganisms such as fungi or the like in the interior. In this regard, embodiments made of plastic film are in particular suitable. In this connection, however, an exchange of oxygen and, in return, carbon dioxide must be made possible for culturing without enabling penetration of undesirable bacteria or other germs. The plastic bag according to the prior art is of a flat arrangement for culturing with a topside perforated as a permeable wall area that is underlaid with a microporous film. The perforated wall area ensures a sufficiently robust wall configuration of the culturing bag and a sufficient permeability through this wall area. The actual filtering function with which gases, for example, oxygen and carbon dioxide, are allowed to pass but undesirable foreign matter and bacteria are kept out is realized by the microporous film.

However, it has been found that the permeability of the microporous film is not well matched to the respective culturing needs. In particular, at the time of starting such a culture in the bag there is often only a minimal oxygen demand and also minimal carbon dioxide release accompanied by great protective needs of the microorganisms with regard to undesirable materials and germs, while after advanced growth of the microorganisms a high gas exchange demand exists accompanied however by a lowered sensitivity of the microorganism culture with regard to extraneous effects.

Accordingly, the object of the invention is thus to design such a culturing bag to be adaptable with regard to barrier function and filtering function of the film wall and to provide a design that with respect to the manufacturing conditions of such culturing bags as well as with regard to handling conditions during the course of culturing is practical but variable.

According to the invention this object is solved by a culturing bag according to the preamble of claim 1 based on the characterizing features of the claim.

It has been found that such a culturing bag with an exterior microporous film that is to be seal-tightly connected at the edges relative to the remaining bag can be imparted with an improved gas exchange as soon as this is required by tearing open or by tearing off the film. In particular, the bag after starting a microorganism culture can first be sealed more tightly with regard to the desired protection relative to drying out and relative to foreign germs in comparison to when the culture has developed.

Tearing open and tearing off can be achieved by an exterior layer of film that is easy to tear as a result of its manufacture, for example, as a polyolefin film that is highly filled with chalk and greatly stretched and therefore can be easily torn, or that as a nonwoven, in a more compacted crosslinked form having micropores, is provided with weakened lines as “predetermined tear locations”. Also, any other finely perforated film with high tear propagation or minimal tear resistance can be used in this connection.

Also, the film, in particular when it appears to be too tear-resistant for practical handling, can be fused or glued with a weak peelable edge seam so that by unseaming the seam it can be torn open or torn off. In general, it is sufficient for improved gas exchange to break open the exterior layer so that the exterior layer must not be completely removed. A complete removal of the exterior layer can however be provided alternatively.

When tearing open the microporous films as well as when tearing open the seam, a pull thread or several thereof can be provided as well as a pull tab of the microporous film that projects past the edge seam for easy handling.

Preferably, directly or indirectly under the exterior layer of microporous film in the permeable wall area the otherwise provided—robust—plastic film of the culturing bag is present that is embodied as an impermeable film mostly in the exterior area and rear area. However, permeability must be ensured here. This can be achieved in a fine-porous form with an appropriate perforation. However, a relatively coarse hole-perforation or needling can be used that is then covered for the filtering function by a fine-porous film, in particular microporous film, underneath the exterior layer. This bottom layer can then be attached also on the inner side of the perforated or needled wall film. Also, it is possible to stack additional films for a step-wise regulation of gas permeability. Expediently, the exterior layer and the next film(s) can be provided with different pore widths that increase from the exterior to the interior. By breaking open the layers from the exterior a higher gas permeability can be achieved in a stepwise fashion.

A two-layer configuration of the permeable wall area with a perforated film wall and only one exterior layer of microporous film is suitable in particular when the special protective and filtering function of the exterior layer is required only for a limited period of time after the start-up phase of the microorganism culture.

A microporous film that is easy to tear and has a predetermined pore width can be realized, for example, by means of a chalk-filled polyolefin film, preferably a polypropylene film optionally also a polyethylene film. It must be stretched so much that the additive during stretching will create pores in the film. The pore size is adjustable by means of the stretching ratio. Such polyolefin films can be connected well in combination with polyolefin bag films by sealing and facilitate segregated recycling.

Films with very small pores are producible also from random orientation layers of a nonwoven material of plastic material by compression and linking. Also, fine-needled or fine-perforated films are suitable. In any case it is important that the material is moisture-resistant, microorganism-resistant and non-rotting. The film must generally be sterilizable as the remaining film of such a culturing bag, for example, by gamma radiation or by gas treatment with ethylene oxide.

Inasmuch as the employed film is a highly tear-resistant nonwoven or another fine-perforated film of high strength, weakened lines can or must facilitate the action of tearing open. However, opening of the exterior layer can also be done along the seam that, for example, can be designed as a sealed seam or peeling seam to be easily torn. Also, a bond seam can be designed to be sufficiently seal-tight but peelable. As a result, a culturing bag can thus be designed such that in the start-up phase of the microorganisms culture that is started in the bag the culturing bag provides a more seal-tight enclosure toward the exterior in accordance with the minimal demand with regard to oxygen supply from the exterior and minimal release of carbon dioxide thus counteracting drying and preventing undesirable penetration of microorganisms. After the culture has developed and propagated and generally also has become less sensitive, the exterior layer is opened in accordance with the higher ventilation demand. Underneath, there can still be provided a microporous film, preferably with greater pore width and therefore a greater permeability. However, in the simplest case a perforated film wall alone can act as a filter.

Embodiments of the invention are illustrated in the drawings and will be explained in the following in more detail. The drawing shows in:

FIG. 1 a schematic view of a culturing bag in a first embodiment;

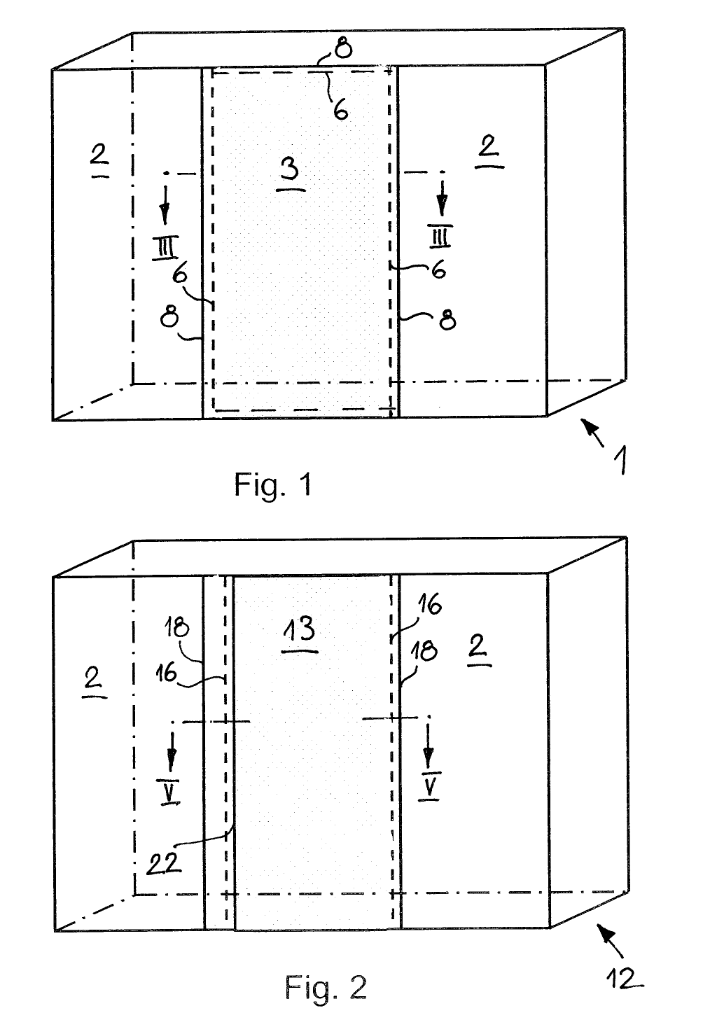

FIG. 2 a schematic view of a culturing bag in a second embodiment;

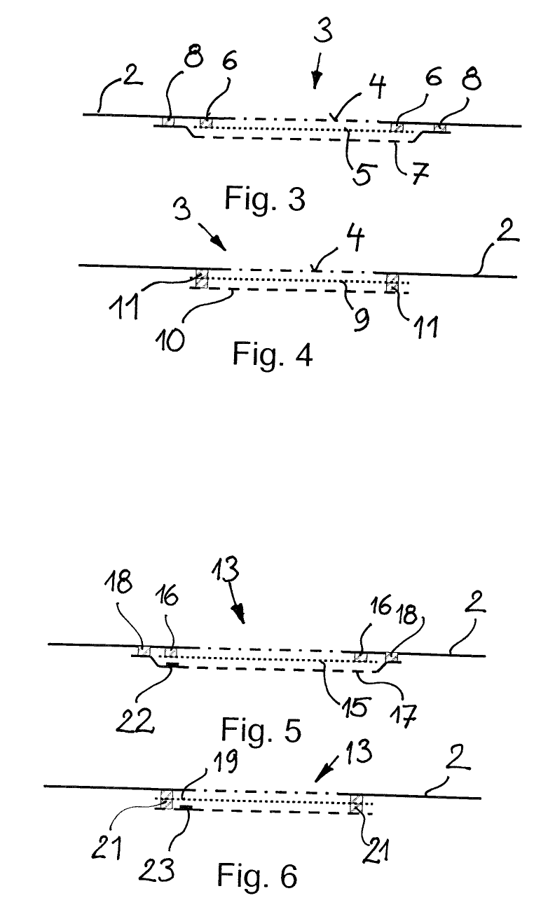

FIG. 3 a section according to line III-III of FIG. 1;

FIG. 4 a section according to FIG. 3 of a modified embodiment;

FIG. 5 a section along V-V of FIG. 2; and

FIG. 6 a section according to FIG. 5 of a modified embodiment.

The culturing bag which is referenced in FIG. 1 with 1 is shown in a parallelepipedal shape, intentionally shown schematically, because it can be designed in any suitable shape, for example, as a simple flat bag, comprised only of two rectangular flush film pieces that are connected to one another by a circumferential edge seam, as an uprightly positionable flat bottom bag or as a gusseted bag, also basically positionable upright and approximating a parallelepipedal shape in the filled state. The bag 1 has a wall 2 with a wall area 3 that is permeable for gases such as oxygen or carbon dioxide. As can be seen in FIG. 3, the wall 2 is provided with a perforation 4 within wall area 3. The wall is formed of a moisture-resistant and non-rotting plastic film. The perforation provided therein in the present case is generated by needling that would allow passage not only of gases but also moisture, germs or optionally dirt when not covered by additional filter layers.

In this regard, for covering the perforation 4 a microporous film 5 of plastic material is placed on top of the perforation and is attached on all sides by a seal-tight sealed seam or bond seam to the film 2. A further microporous film 7 covering the microporous film 5 is provided on the exterior side of the culturing bag 1 and is secured with its own edge seam 8 at the edges seal-tightly to the film of the wall 2. By means of the microporous films 5 and 7 germs and dirt cannot penetrate the permeable wall area above the perforation 4 but exchange of gases through the microporous films 5 and 7 is possible. The films used in this connection are plastic films, in particular polyolefin films wherein for a possibly required steam sterilization polypropylene is preferred; otherwise, polyethylene film is also suitable. The thickness of the wall film is set to be between 30 and 150 mm depending on the predetermined filling material and in particular the filled-in weight.

The filter function is assigned to the microporous films 5 and 7 that are comprised, for example, of a gas-permeable polypropylene film that is filled innately with a high proportion of chalk and is then stretched to such an extent that micropores are produced. In this regard, the exterior film 7 can have finer pores in comparison to the film 5 positioned underneath. This is achievable in that the pore size can be affected by different degrees of stretching.

When such a culturing bag after sterilization is filled with nutrient material that is inoculated with a mycelium, the desired microorganisms culture can develop with gas exchange without dirt, germs and moisture being able to penetrate and without drying up. As soon as the culture has developed more strongly, the breathing demand is however also greater. For this purpose, a greater gas permeability in the area of the perforation is to be provided.

For this purpose, the microporous film 7 that may indeed be splitable can be torn open so that gas exchange must penetrate only a filter barrier in the form of the microporous film 5. When the latter is provided with greater micropores in comparison to the initially externally located film 7 a further enhancement of the gas exchange is provided.

The film 5 can also be torn open or torn off when the culture has developed so strongly or so robustly that foreign germs no longer must be kept away. The perforation 4 in the wall film 2 however still has to fulfill the task of preventing the cultures or their substrate material from escaping.

An opening of the microporous films can also be enabled at the seam 8 or the seam 6 when they are designed with known means as weakened sealed seams. Bond seams are to be designed to be peelable by using known adhesives. The design according to FIG. 3 with separately extending edge seams 6 and 8 makes it possible to unseam the seams independently from one another, i.e., the microporous films 5 and 7 can be lifted off sequentially from the exterior to the interior.

An alternative embodiment according to FIG. 2 also provides a wall 2 of a wall film with a perforation area 4 in which two microporous films 9, 10 stacked on top one another are connected by a common edge seam 11 seal-tightly to the wall film 2. This embodiment is in particular advantageous in regard to manufacturing considerations for a common sealing action wherein the films preferably are embodied to easily split for tearing them open.

In regard to the disclosure above it is understood that the inwardly positioned microporous film 5 or 9, when not provided also for being torn open, can be attached to the other inwardly positioned sides of the wall film 2.

The embodiment of a culturing bag 12 supplemented with details according to FIG. 2 is also comprised of film that is joined to a container in any shape wherein in one wall 2 a wall area 13 is formed that by perforation, needling, or slitting provides a relatively coarse filter that, according to the section illustration of FIG. 5 or 6 is covered by microporous films 15, 17 or 19, 20 in such a way that a protection with regard to foreign germs, contaminants and the like is provided but a gas exchange is still possible. These microporous films 15, 17, 19, 20 are also connected by adjacently extending seal-tight edge seams 16 and 18 and a common edge seam 21 to the wall 2. For facilitating the tearing action an opening aid in the form of a pull thread 22 or 23 is provided that, in its longitudinal direction, can extend past the seam 18 or 21 in order to facilitate gripping by hand. By means of such threads 22, 23 that can extend straight or curved or angled the tearing action is facilitated. In case of microporous films that are innately less easily torn, e.g., needled films or compacted microporous nonwoven films, it is expedient to provide as an opening aid in addition to the pull thread a weakened line or several thereof. Also, the microporous film can project past the edge seam to form a pull tab or a gripping area as an opening aid.

In the illustrated case the wall area 3 that is covered by microporous film is illustrated as a partial area of the wall 2. It is understood, of course, that such a wall area can be designed to be larger as needed, for example, so as to extend across the entire surface area of the wall 2. Even greater breathing surfaces can be provided in that the microporous film is also provided on the back side of the bag. In principle, a culturing bag can be enclosed as a whole with one or with several layers of microporous film and can be held together by a seal-tight edge seam.

What is claimed is:

1.-13. (canceled)

14. A culturing bag for microorganisms, the culturing bag comprising:
a wall formed of plastic film wherein at least one wall area of the wall is permeable for oxygen and carbon dioxide;
a first layer of microporous film attached to the at least one wall area as an exterior layer so as to form a multi-layer configuration in the at least one wall area;
wherein the first layer of microporous film is sealed by a seal-tight edge seam to the wall;
wherein the microporous film is adapted to be torn open or torn off.

15. The culturing bag according to claim 14, wherein the microporous film is designed to be splitable.

16. The culturing bag according to claim 15, wherein the microporous film is provided with at least one opening aid.

17. The culturing bag according to claim 15, wherein the microporous film is provided with at least one pull thread.

18. The culturing bag according to claim 14, wherein the microporous film is provided with at least one weakened line for tearing the microporous film.

19. The culturing bag according to claim 14, wherein the edge seam that attaches the microporous film to the wall is adapted to be unseamed at least in some areas.

20. The culturing bag according to claim 19, wherein the microporous film at least partially projects with a grip area past the edge seam.

21. The culturing bag according to claim 14, further comprising at least one second layer of microporous film arranged between the first layer of microporous film and the wall.

22. The culturing bag according to claim 21, wherein the at least one second layer of microporous film is adapted to be torn open or torn off.

23. The culturing bag according to claim 21, wherein the at least one second layer of microporous film has pores that are greater than pores of the first layer of microporous film.

24. The culturing bag according to claim 21, wherein the at least one wall area where the first layer of microporous film and the at least one second layer of microporous film are arranged is a continuous but perforated film area of the wall.

25. The culturing bag according to claim 14, wherein the microporous film is comprised of a chalk-filled and highly-stretched polyolefin film.

26. The culturing bag according to claim 14, wherein the microporous film is comprised of a nonwoven or a nonwoven-like structure.

27. The culturing bag according to claim 14, wherein the microporous film is comprised of a fine-perforated or hole-perforated plastic film.
